(19) United States
(12) Patent Application Publication
(10) Pub. No.: US 2022/0290151 A1
(43) Pub. Date: Sep. 15, 2022

(54) USE OF MÜLLERIAN INHIBITING SUBSTANCE INHIBITORS FOR TREATING CANCER

(71) Applicants: INSERM (INSTITUT NATIONAL DE LA SANTÉ ET DE LA RECHERCHE MÉDICALE), Paris (FR); INSTITUT RÉGIONAL DU CANCER DE MONTPELLIER, Montpellier (FR); UNIVERSITÉ DE MONTPELLIER, Montpellier (FR)

(72) Inventors: Andre PELEGRIN, Montpellier (FR); Thierry CHARDES, Montpellier (FR); Isabelle NAVARRO-TEULON, Montpellier (FR); Maeva CHAUVIN, Montpellier (FR)

(21) Appl. No.: 17/635,894

(22) PCT Filed: Sep. 25, 2020
(86) PCT No.: PCT/EP2020/076904

§ 371 Date: Feb. 16, 2022

(30) Foreign Application Priority Data
Sep. 27, 2019 (EP) 19306215.5

(51) Int. Cl. C12N 15/113; C12N 15/115; A61P 35/00

(52) U.S. Cl. CPC C12N 15/1136; C12N 15/115; A61P 35/00; C12N 2310/14; C12N 2310/11

(57) ABSTRACT

In ovarian carcinoma, Müllerian Inhibiting Substance (MIS) type II receptor (MISRII) and the MIS/MISRII signaling pathway are potential therapeutic targets. Conversely, the role of the three MIS type I receptors (MISRI; ALK2, ALK3 and ALK6) in this cancer needs to be clarified. Using four ovarian cancer cell lines and ovarian cancer cells isolated from patients' tumor ascites, the inventors found that ALK2 and ALK3 are the two main MISRIs involved in MIS signaling at low and high MIS concentrations, respectively. Moreover, high MIS concentrations were associated with apoptosis and decreased clonogenic survival, whereas low MIS concentrations improved cancer cell viability. Finally, the inventors showed that MIS siRNA inhibited MIS pro-survival effect. These last results open the way to an innovative therapeutic approach to suppress MIS proliferative effect, instead of administering high doses of MIS to induce cancer cell apoptosis.

### FIELD OF THE INVENTION

The present invention relates to a müllerian inhibiting substance (MIS) inhibitor for use in the treatment of MIS or MISRII positive cancer in a subject in need thereof.

### BACKGROUND OF THE INVENTION

Müllerian Inhibiting Substance (MIS) is a member of the TGFβ family, and acts by binding to its specific receptor (MIS type II receptor; MISRII) that recruits type I receptors (MISRI: ALK2, ALK3 and ALK6). MISRI phosphorylation induces SMAD 1/5/8 phosphorylation and their migration into the nucleus where through SMAD4, they regulate different responsive genes, depending on the target tissue (di Clemente et al., 2010; Josso and Clemente, 2003). Preclinical in vitro and in vivo findings as well as data from clinical samples (Bakkum-Gamez et al., 2008; Masiakos et al., 1999; Meirelles et al., 2012; Pépin et al., 2015; Renaud et al., 2005; Wei et al., 2010) have demonstrated that MISRII and the MIS/MISRII signaling pathway are potential therapeutic targets in gynecological tumors, and particularly in ovarian carcinoma (reviewed in (Kim et al., 2014)). Moreover, Beck T N et al. showed that in lung cancer, MIS/MISRII signaling regulates epithelial-mesenchymal transition (EMT) and promotes cell survival/proliferation (Beck et al., 2016). They suggested that MIS/MISRII signaling role in EMT regulation was important for chemoresistance. Furthermore, the MIS/MISRII signaling pathway has recently been shown to be implicated in colorectal cancers in which (i) the MIS gene is upregulated (Pellatt et al., 2018), and (ii) high MIS RNA expression is an unfavorable prognostic factor (n=597 patients with a follow-up of more than 12 years) (Uhlen et al., 2017). This signaling cascade could be targeted using recombinant MIS or anti-MISRII antibodies. However, the use of recombinant MIS has been hampered by the difficulties linked to the production of sufficient amounts of bioactive MIS and to its delivery at the tumor site (Donahoe et al., 2003). Recently, Pépin et al. described an original production strategy and an alternative delivery approach using gene therapy (not yet in clinical phase) (Pépin et al., 2013, 2015). Among anti-MISRII antibodies (Salhi et al., 2004) and antibody fragments (Yuan et al., 2006, 2008), the monoclonal antibody (MAb) 12G4 and its humanized version have been extensively evaluated in preclinical studies (Bougherara et al., 2017; Estupina et al., 2017; Gill et al., 2017; Kersual et al., 2014), and the humanized antibody (GM-102 or murlentamab) is now tested in clinical trials (NCT02978755, NCT03799731). The mechanism of action of the glyco-engineered murlentamab involves antibody-dependent cell-mediated cytotoxicity and antibody-dependent cell phagocytosis, but almost no apoptosis, suggesting that the effect is not directly related to the MIS signaling pathway (Bougherara et al., 2017; Estupina et al., 2017). Indeed, in MISRII-positive cancer cells, MIS inhibits proliferation and induces apoptosis.

To understand why the MIS signaling pathway is not implicated in the mechanisms of action of this anti-MISRII MAb, the inventors analyzed the role of the three MISRI (ALK2, ALK3 and ALK6) in ovarian carcinoma cell lines and carcinoma cells isolated from ascites samples of patients with ovarian carcinoma. Indeed, although ALK2, ALK3 and ALK6 roles in several cell types have been studied during development and in other physiological conditions (Belville et al., 2005; Clarke et al., 2001; Josso et al., 1998; Orvis et al., 2008; Sèdes et al., 2013; Visser et al., 2001; Zhan et al., 2006), few data are available in cancer. Basal et al. demonstrated that MISRII, ALK2, ALK3 and ALK6 are expressed in epithelial ovarian cancer (immunohistochemistry analysis of 262 samples), but did not assess their specific role (Basal et al., 2016).

Herein, the inventors found that ALK2 and ALK3 are the two main MISRI used for MIS signaling in four ovarian cancer cell lines (derived from two epithelial ovarian tumors and from two sex cord-stromal tumors, including one granulosa cell tumor), and that they have a differential role according to MIS concentration. They then showed that cancer cell viability promotion by MIS at low concentration (below 0.5 to 13 nM) can be inhibited using MIS siRNAs. This observation opens the way to an innovative therapeutic approach to suppress MIS proliferative effect, instead of administering high doses of MIS to induce apoptosis.

### SUMMARY OF THE INVENTION

In ovarian carcinoma, Müllerian Inhibiting Substance (MIS) type II receptor (MISRII) and the MIS/MISRII signaling pathway are potential therapeutic targets. Using four ovarian cancer cell lines and ovarian cancer cells isolated from patients' tumor ascites, the inventors found that ALK2 and ALK3 are the two main MISRIs involved in MIS signaling at low and high MIS concentrations, respectively. Moreover, high MIS concentrations were associated with apoptosis and decreased clonogenic survival, whereas low MIS concentrations improved cancer cell viability. Finally, the inventors showed that MIS siRNA inhibited MIS pro-survival effect. These last results open the way to an innovative therapeutic approach to suppress MIS proliferative effect, instead of administering high doses of MIS to induce cancer cell apoptosis.

Thus the present invention relates to a müllerian inhibiting substance (MIS) inhibitor for use in the treatment of MIS or MISRII positive cancer in a subject in need thereof. More particularly, the invention is defined by its claims.

### DETAILED DESCRIPTION OF THE INVENTION

### Therapeutic Methods and Uses

A first aspect of the invention relates to a müllerian inhibiting substance (MIS) inhibitor for use in the treatment of MIS or MISRII positive cancer in a subject in need thereof.

Thus, the invention relates to a müllerian inhibiting substance (MIS) inhibitor for use in the treatment of MIS or MISRII positive cancer in a subject in need thereof, wherein the cancer is selected from the group consisting of gynecological cancer, lung cancer or colorectal cancer.

In particular, the MIS or MISRII positive cancer is a gynecological cancer, lung cancer or colorectal cancer.

In other word, the invention refers to a method of treating gynecological cancer, lung cancer or colorectal cancer in a subject in need thereof, comprising administrating to said subject a therapeutically effective amount of a MIS inhibitor.

As used herein, the term “subject” refers to any mammal, such as rodent, a feline, a canine, a primate or human. In some embodiment of the invention, the subject refers to any subject afflicted with or susceptible to be afflicted with MIS or MISRII positive cancer. Particularly, in preferred embodiment, the subject is a human afflicted with or susceptible to be afflicted with gynecological cancer, lung cancer or colorectal cancer.

In some embodiment, the subject is a human afflicted with or susceptible to be afflicted with ovarian cancer.

As used herein, the term “treatment” or “treating” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein “the MIS or MISRII positive cancer” refers to cancer which express the MIS. In some embodiment, the MIS or MISRII positive cancer is selected from the group consisting of breast cancer, prostate cancer, lung cancer, colorectal cancer, or gynecological cancer (see Kim et al, 2014).

As used herein, the term “lung cancer”, also known as “lung carcinoma” includes the well-accepted medical definition that defines lung cancer as a medical condition characterized by uncontrolled cell growth in tissues of the lung. The main types of lung cancer are lung carcinoid tumor, small-cell lung carcinoma (SCLC) and non-small-cell lung carcinoma (NSCLC) such as squamous cell carcinoma, adenocarcinoma, and large cell carcinoma. Additionally, the term “lung cancer” includes all types of lung cancer at all stages of progression. The staging system most often used for lung cancer is the American Joint Committee on Cancer (AJCC) TNM system which is based on the size of the tumor, the spread to nearby lymph nodes and the spread (metastasis) to distant sites.

As used herein, the term “colorectal cancer” or “CRC” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions, which are characterized by cancer of cells of the duodenum and small intestine (jejunum and ileum). Additionally, the term “colorectal cancer” includes all types of colorectal cancer at all stages of progression. The earliest stage colorectal cancers are called stage 0 (a very early and superficial cancer), and then range from stage I through IV. In stage IV of colorectal cancer, also known as metastatic colorectal, the cancer has spread beyond the colon or rectum to distant organs, such as the liver or lungs. The staging system most often used for CRC is the American Joint Committee on Cancer (AJCC) TNM system which is based on the size of the tumor, the spread to nearby lymph nodes and the spread (metastasis) to distant sites.

As used herein, the term “gynecological cancer” has its general meaning in the art and refers to cancer that develop in woman's reproductive tract. The types of gynecological cancers are cervical cancer, uterine cancer also known as womb cancer or endometrial cancer, ovarian cancer, vaginal cancer, vulvar cancer, primary peritoneal cancer, gestational trophoblastic disease and fallopian tube cancer. Cervical cancer occurs when the cells of the cervix grow abnormally and invade other tissues and organs of the body and include squamous cell carcinoma; adenocarcinoma; adenosquamous carcinoma; small cell carcinoma: neuroendocrine tumor; glassy cell carcinoma; villoglandular adenocarcinoma; cervical melanoma and cervical lymphoma. Uterine refer to any types of cancer which occur in the uterus and include endometrial carcinoma such as endometrial adenocarcinoma, endometrial adenosquamous carcinoma, papillary serous carcinoma, uterine clear-cell carcinoma, mucinous carcinoma of endometrium, mucinous adenocarcinoma of endometrium and endometrial squamous cell carcinoma; transitional cell carcinoma of the endometrium; endometrial stromal sarcomas; malignant mixed müllerian tumors; uterine fibroma; and uterine sarcoma such as uterine carcinosarcoma, uterine adenosarcoma and uterine leiomyosarcomas. Vaginal cancer is a rare cancer occurring in vagina and include vaginal squamous cell carcinoma; vaginal melanoma; and vaginal sarcoma. Vulvar cancer is a type of cancer that occurs on the outer surface area of the female genitalia and include vulvar squamous cell carcinoma; vulvar melanoma; vulvar basal cell carcinoma; Bartholin gland carcinoma; vulvar adenocarcinoma and vulvar sarcoma. Ovarian cancer is a cancer that forms in or on an ovary and include: ovarian epithelial tumors such as ovarian mucinous carcinoma, high-grade serous carcinoma, ovarian endometrioid carcinoma, ovarian clear-cell carcinoma, ovarian low malignant potential tumors and primary peritoneal carcinoma; germ cell tumors such as teratomas, dysgerminoma ovarian germ cell cancer, choriocarcinoma tumors and endodermal sinus tumors; sex-cord stromal tumors such as granulosa cell tumors, granulosa-theca tumors, ovarian fibroma, leydic cell tumors, sertoli cell tumors, sertoli-leydig tumors and gynandroblastoma; ovarian sarcoma such as ovarian carcinosarcomas, ovarian adenosarcomas, ovarian leiomyosarcomas and ovarian fibrosarcomas; krukenberg tumors; and ovarian cysts.

In some embodiment, the cancer is a gynecological cancer.

In some embodiment, the cancer is an ovarian cancer.

As used herein, a “therapeutically effective amount” is intended for a minimal amount of active agent which is necessary to impart therapeutic benefit to a patient. For example, a “therapeutically effective amount of the active agent” to a patient is an amount of the active agent that induces, ameliorates or causes an improvement in the pathological symptoms, disease progression, or physical conditions associated with the disease affecting the patient.

As used herein the terms “administering” or “administration” refer to the act of injecting or otherwise physically delivering a substance as it exists outside the body (e.g., an inhibitor of MIS) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

As used herein, the term “müllerian inhibiting substance” or “MIS”, also known as “anti-müllerian hormone” or “AMH”, has its general meaning in the art and refers to a glycoprotein hormone structurally related to inhibin and activin from the transforming growth factor beta (TGFβ) superfamily, with key roles in growth differentiation and folliculogenesis. MIS is a 140 kDa dimeric glycoprotein that is encoded by AMH gene on human chromosome 19p13.3. Its entrez reference is 268 and its Uniprot reference is P03971. The MIS acts by binding to its specific MIS type II receptor (MISRII or AMHR2) that recruits type I receptor (MISRI or AMHR1). ALK2, ALK3 and ALK6 are the three variants of MISRI. The phosphorylation of MISRI induces SMAD 1/5/8 phosphorylation and regulate different responsive gene, depending on the target tissue, through SMAD4.

According to the invention, the müllerian inhibiting substance (MIS) inhibitor can be a MIS expression inhibitor or a MIS activity inhibitor.

In some embodiment, the MIS inhibitor for use according to the invention is a MIS activity inhibitor such as an antibody, a peptide, a polypeptide, an aptamer or a MIS expression inhibitor such as antisense oligonucleotides or siRNA.

Thus, the invention refers to a müllerian inhibiting substance inhibitor for use in the treatment of MIS or MISRII positive cancer in subject in need thereof, wherein said inhibitor is a MIS activity inhibitor such as an antibody, a peptide, a polypeptide, an aptamer or a MIS expression inhibitor such as antisense oligonucleotides or siRNA.

In particular embodiment, the müllerian inhibiting substance inhibitor blocks the recruiting of MIS type I receptor MISRI (i.e ALK2, ALK3 or ALK6) by the complex MISRII/MIS.

The term “MIS expression inhibitor” denotes inhibitors of the expression of the gene coding for MIS. Thus, the term “MIS expression inhibitor” refers to a natural or synthetic compound that has a biological effect to inhibit the expression of the MIS gene.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs which are modified, by processes such as capping, polyadenylation, methylation, and editing, and proteins (e.g., phosphatidylserine receptor) modified by, for example, methylation, acetylation, phosphorylation, ubiquitination, SUMOylation, ADP-ribosylation, myristilation, and glycosylation.

MIS expression inhibitor for use in the present invention may be based on antisense oligonucleotide constructs. Anti-sense oligonucleotides, including anti-sense RNA molecules and anti-sense DNA molecules, would act to directly block the translation of MIS mRNA by binding thereto and thus preventing protein translation or increasing mRNA degradation, thus decreasing the level of MIS, and thus activity, in a cell. For example, antisense oligonucleotides of at least about 15 bases and complementary to unique regions of the mRNA transcript sequence encoding MIS can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as MIS expression inhibitor for use in the present invention. MIS gene expression can be reduced by contacting the subject or cell with a small double stranded RNA (dsRNA), or a vector or construct causing the production of a small double stranded RNA, such that MIS expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Both antisense oligonucleotides and siRNAs useful as MIS expression inhibitor can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides and siRNAs of the invention may be delivered in vivo alone or in association with a vector. In its broadest sense, a “vector” is any vehicle capable of facilitating the transfer of the antisense oligonucleotide or siRNA nucleic acid to the cells and preferably cells expressing MIS. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide or siRNA nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intravenous, intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

As used herein the term “müllerian inhibiting substance (MIS) activity inhibitor” refers to any compound able to inhibit MIS activity and selectively blocks or inactivates MIS or any compound which destabilize MIS. Thus, the term “müllerian inhibiting substance (MIS) activity inhibitor” refers to compounds that bind or target MIS. The “müllerian inhibiting substance (MIS) activity inhibitor” refers to compounds that block MIS interaction with its specific receptor, the MIS type II receptor and thus inhibits the MISRII/MIS signalling pathways. The term “müllerian inhibiting substance (MIS) activity inhibitor” also relates to compounds that block the recruiting of MIS type I receptor MISRI (i.e ALK2, ALK3 or ALK6) by the complex MISRII/MIS. Typically, an activity inhibitor of müllerian inhibiting substance (MIS) is an antibody, a small organic molecule, a peptide, a polypeptide or an aptamer.

In one embodiment, the MIS activity inhibitor is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S.D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against MIS of the invention as above described, the skilled man in the art can easily select those inhibiting MIS.

In one embodiment, the MIS activity inhibitor is a small organic molecule.

The term “small organic molecule” refers to a low molecular weight compound, e.g a molecule (natural or not) of a size comparable to those organic molecules generally used in pharmaceuticals. The term excludes biological macromolecules (e. g., proteins, nucleic acids, etc.). Preferred small organic molecules range in size up to about 10000 Da, more preferably up to 5000 Da, more preferably up to 2000 Da and most preferably up to about 1000 Da.

In another embodiment, the MIS activity inhibitor is an anti-MIS antibody (the term including “antibody portion”).

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a human antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of MIS. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the antigen may be provided as synthetic peptides corresponding to antigenic regions of interest in MIS. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3 A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4. In a preferred embodiment, the MIS activity inhibitor of the invention is a Human IgG4.

In another embodiment, the anti-MIS antibody for use according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In one embodiment, the MIS activity inhibitor is a polypeptide.

In particular embodiment, the polypeptide is an antagonist of MIS and is capable to prevent the function of MIS.

In one embodiment, the polypeptide of the invention may be linked to a cell-penetrating peptide” to allow the penetration of the polypeptide in the cell.

The term “cell-penetrating peptides” are well known in the art and refers to cell permeable sequence or membranous penetrating sequence such as penetratin, TAT mitochondrial penetrating sequence and compounds (Bechara and Sagan, 2013; Jones and Sayers, 2012; Khafagy el and Morishita, 2012; Malhi and Murthy, 2012).

The polypeptides of the invention may be produced by any suitable means, as will be apparent to those of skill in the art. In order to produce sufficient amounts of polypeptide or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

The MIS inhibitor for use according to the invention can be administered in combination with a classical treatment of MIS or MISRII positive cancer.

Thus, the invention also refers to i) a müllerian inhibiting substance (MIS) inhibitor and ii) a classical treatment of MIS or MISRII positive cancer for use in the treatment of MIS or MISRII positive cancer in a subject in need thereof.

In other word, the invention refers to a method of treating MIS or MISRII positive cancer in a subject in need thereof, comprising administrating to said subject a therapeutically effective amount of a MIS inhibitor and a classical treatment of MIS or MISRII positive cancer.

As used herein, the term “classical treatment” refers to any compound, natural or synthetic, used for the treatment of MIS or MISRII positive cancer.

In a particular embodiment, the classical treatment refers to radiation therapy, immunotherapy or chemotherapy.

According to the invention, compound used for the classical treatment of MIS or MISRII positive cancer may be selected in the group consisting in: EGFR inhibitor such as cetuximab, panitumumab, bevacizumab and ramucirumab; kinase inhibitor such as erlotinib, gefitinib afatinib, regorafenib and larotrectinib; immune checkpoint inhibitor; chemotherapeutic agent and radiotherapeutics agent.

As used herein, the term “chemotherapy” refers to cancer treatment that uses one or more chemotherapeutic agents.

As used herein, the term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a camptothecin (including the synthetic analogue topotecan and irinotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl.

Ed. Engl. 33: 183-186 (1994); dynemicin, including dynemicin A; an esperamicin; as well as neocarzinostatin chromophore and related chromoprotein enediyne antibiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, trifluridine, tipiracil, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum such as oxaliplatin, cisplatin and carbloplatin; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; ziv-aflibercept; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type

As used herein, the term “radiation therapy” has its general meaning in the art and refers the treatment of MIS or MISRII positive cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a colorectal cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

As used herein, the term “immune checkpoint inhibitor” refers to molecules that totally or partially reduce, inhibit, interfere with or modulate one or more immune checkpoint proteins.

As used herein, the term “immune checkpoint protein” has its general meaning in the art and refers to a molecule that is expressed by T cells in that either turn up a signal (stimulatory checkpoint molecules) or turn down a signal (inhibitory checkpoint molecules).

Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, PD-L1, LAG-3, TIM-3 and VISTA.

According to the invention, the MIS inhibitor and the classical treatment can be used as a combined treatment.

As used herein, the terms “combined treatment”, “combined therapy” or “therapy combination” refer to a treatment that uses more than one medication. The combined therapy may be dual therapy or bi-therapy. The medications used in the combined treatment according to the invention are administered to the subject simultaneously, separately or sequentially.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

### Pharmaceutical Composition

The MIS inhibitor of the invention may be used or prepared in a pharmaceutical composition.

In one embodiment, the invention relates to a pharmaceutical composition comprising the MIS inhibitor of the invention and a pharmaceutical acceptable carrier for use in the treatment of MIS or MISRII positive cancer in a subject of need thereof.

In some embodiment, the MIS or MISRII positive cancer is selected from the group consisting of gynecological cancer, lung cancer or colorectal cancer.

Typically, the inhibitor of the invention may be combined with pharmaceutically acceptable excipients, and optionally sustained-release matrices, such as biodegradable polymers, to form therapeutic compositions.

As used herein, the term “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising inhibitors of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In addition to the MIS inhibitors of the invention formulated for parenteral administration, such as intravenous or intramuscular injection, other pharmaceutically acceptable forms include, e.g. tablets or other solids for oral administration; liposomal formulations; time release capsules; and any other form currently used.

Pharmaceutical compositions of the invention may include any further active agent which is used in the treatment of MIS or MISRII positive cancer.

In one embodiment, said additional active agents may be contained in the same composition or administrated separately.

In another embodiment, the pharmaceutical composition of the invention relates to combined preparation for simultaneous, separate or sequential use in the treatment of MIS or MISRII positive cancer.

In some embodiment, the MIS or MISRII positive cancer is selected from the group consisting of gynecological cancer, lung cancer or colorectal cancer.

The invention also provides kits comprising the MIS inhibitor of the invention. Kits containing the MIS inhibitor of the invention find use in therapeutic methods.

The invention will be further illustrated by the following figures and examples. However, these examples and figures should not be interpreted in any way as limiting the scope of the present invention.

### FIGURES

FIG. 1: Graphical abstract of the paradoxical effect of müllerian inhibiting substance (MIS) in ovarian carcinomas and of the proposed therapeutic strategy of MIS inhibition.

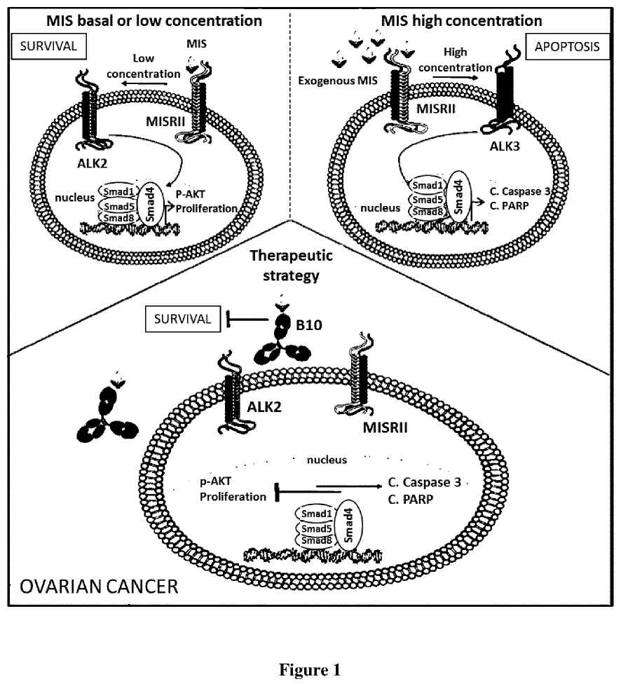

FIG. 2: Recombinant MIS (LRMIS) induces MIS signaling in COV434-MISRII and SKOV3-MISRII cells. A. Incubation with 1.6 to 25 nM LRMIS for 6 hours promotes apoptosis (caspase 3/7 activity). B. Clonogenic survival was quantified after culture in the presence of 1.6 to 25 nM LRMIS for 11 days by direct clone counting (COV434-MISRII cells) or by estimating the number of clones by OD at 595 nm after cell lysis (SKOV3-MISRII cells).

FIG. 3: Involvement of ALK2, ALK3 and ALK6 in MIS effect in COV434-MISRII and SKOV3-MISRII cells. Apoptis initiation (caspase 3/7 activity) was analyzed after incubation of siALK2, siALK3 or siALK6 transfected COV434-MISRII or SKOV3-MISRII cells with 25 nM MIS for 6 hours (started 48 hours after siRNA transfection).

FIG. 4: Low-dose recombinant MIS (LRMIS) promotes cell viability in COV434-MISRII, SKOV3-MISRII, OVCAR8 and KGN cells. A. Cell viability (MTS assay) after incubation with 0.8 to 25 nM LRMIS for 3 days. B. Effect of siRNA-mediated MIS silencing on cell viability at days 3 post-transfection of siRNAs against MIS.

### EXAMPLE

Material & Methods

Cell Lines

The human COV434 (sex cord-stromal tumor) (Chan-Penebre et al., 2017; Zhang et al., 2000) and KGN (granulosa cell tumor) (Nishi et al., 2001) cell lines were kind gifts from Dr. PI Schrier (Department of Clinical Oncology, Leiden University Medical Center, Nederland) and Dr T Yanase (Kyushu University, Fukuoka, Japan), respectively. The human epithelial ovarian cancer cell lines SKOV3 and NIH-OVCAR8 were from ATCC (ATCC® HTB-77) and from the Division of Cancer Treatment and Diagnosis, NCI, Frederick, Md., USA, respectively. Cells were grown in DMEM F12 medium without red phenol containing 10% heat-inactivated fetal bovine serum (FBS). COV434-MISRII and SKOV3-MISRII cells were supplemented with 0.33 mg/ml geneticin (InvivoGen, ant-gn-1). Cells were grown at 37° C. in a humidified atmosphere with 5% CO2, and medium was replaced twice per week. Cells were harvested with 0.5 mg/ml trypsin/0.2 mg/ml EDTA. All culture media and supplements were purchased from Life Technologies. Inc. (Gibco BRL). The HEK293K cells, used for antibody production by the GenAc platform at IRCM, were grown in DMEM F12 with phenol red containing 10% heat-inactivated FBS.

The COV434-MISRII and SKOV3-MISRII cell lines were generated by transfection of the cDNA encoding full-length human MISRII (Kersual et al., 2014). The cDNA coding for full-length human MISRII in the pCMV6 plasmid was a generous gift by J Teixeira (Pediatric Surgical Research Laboratories, Massachusetts General Hospital, Harvard Medical School). MISRII cDNA was first subcloned in the pcDNA3.1.myc-His vector (Invitrogen) using the EcoRI and XhoI restriction sites (enzymes from New England BioLabs), and then, using the EcoRI and SalI sites, in the pIRES1-EGFP vector, a kind gift from F Poulat (IGH-UPR1142 CNRS). Twenty-four hours before transfection, COV434 cells were seeded in 10 cm cell culture dishes at 80% of confluence. The MISRII construct was transfected using the Fugene transfection kit according to the manufacturer's protocol. After 48 h, transfection medium was replaced with fresh medium containing 0.5 mg/ml geneticin and was then changed twice/week for two weeks. Then, cells were harvested and sorted using a FACSAria cytometer (Becton Dickinson) in 96-well plates. For each cell line, a clone that strongly expressed MISRII was selected and designed as COV434-MISRII and SKOV3-MISRII.

Primary Tumor Cells from Patients' Ascites

Ascites samples from two patients with ovarian cancer were obtained from the “Institut Cancer Montpellier, ICM” according to the French laws and after their informed consent. These two patients were selected because they never received any chemotherapy and were waiting for surgical intervention at the ICM—Val d'Aurelle Hospital. Freshly obtained ascites were aliquoted in 50 ml conical centrifuge tubes and spun at 1300 rpm for 5 min. Cell pellets were re-suspended in ammonium-chloride-potassium buffer (ACK lysis buffer: NH4Cl 150 nM; KHCO3 10 nM; Na2EDTA 0.1 nM) to lyse red blood cells (RBC) on ice for 5 min. The process was repeated until RBC lysis was complete. Then, cell pellets were plated on 150 mm cell culture dishes with 20 ml DMEM F12-Glutamax (Gibco) and 10% FBS. The same day, 100,000 cells were harvested to assess MISRII expression by FACS. Cells were then plated in DMEM F12/10% FBS for 30 minutes to rapidly eliminate adherent fibroblasts (0 Donnell et al., 2014). Non-adherent cells were transferred in new dishes with DMEM F12/10% FBS. Low-passage cells were used for experiments or frozen in liquid nitrogen.

Müllerian Inhibiting Substance (MIS) Production and Assay

The active recombinant MIS (LRMIS), described in the work by D Pépin et al. (Pépin et al., 2013, 2015) was used in our study. It contains (i) the 24AA leader sequence of albumin instead of the MIS leader sequence to increase production and secretion, and (ii) the RARR/S furin/kex2 consensus site instead of the native MIS RAQR/S sequence at position 423-428 to improve cleavage. MIS dosages were performed using the Elecsys® AMH (Anti-Mullerian Hormone) assay from Roche. All experiments involving LRMIS were performed in culture medium containing 1% FBS because bovine MIS can signal through human MISRII (Cate et al., 1986). In these experimental conditions, endogenous MIS concentration ranged from 5 to 10 pM in fresh medium to about 10 to 15 pM after 5 days of cell culture. To determine endogenous MIS concentration in cell culture supernatants, one million cells were plated in 100 mm cell culture dishes in 10 ml DMEM F12/1% FBS. Every 24 h, 300 μl of medium was removed for MIS dosage.

siRNA Transfections and Assays

siRNAs sequences were designed with the Rosetta algorithm and are backed by Sigma-Aldrich predesigned siRNA guarantee. We used a pool of three siRNAs for each ALK receptor and for MIS. Cells were plated in 24-well plates up to 60-80% confluence. Transfection was performed in medium with 1% FBS using Lipofectamine RNAiMax Transfection Reagent diluted in Opti-MEM Medium according to the provider (Thermofisher cat#13778-150). siRNAs were diluted to 300 ng/ml (siRNAs against ALK2, ALK3, and ALK6) and to 1 μg/ml (siRNAs against MIS) in Opti-MEM, and the siRNA-Lipofectamine (1:1) mixture was added to the cells for 6 h. Cells were washed and cultured in DMEM F12/1% FBS. Experiments with siRNA-transfected cells were performed at 24 h (COV434-MISRII cells) and 48 h (SKOV3-MISRII cells) after transfection.

Western Blot Analysis

Cells were washed with PBS and scrapped immediately in RIPA lysis buffer (Santa Cruz) that included 200 mM PMSF solution, 100 mM sodium orthovanadate solution, and protease inhibitor cocktail. The protein concentration was determined using the BCA assay protein quantitation kit (Interchim). Cell extracts were heated at 95° C. for 5 min, separated (50 μg proteins/well) on 10% SDS-PAGE in reducing conditions (5% 2β-mercaptoethanol), and transferred to PVDF membranes (Biorad). Membranes were saturated in Tris-buffered saline, containing 0.1% Tween 20 and 5% non-fat dry milk, and probed with the relevant primary antibodies at RT for 1 h. After washing, peroxidase-conjugated IgG secondary antibodies were added (1/10,000) at RT for 1 h. After washing, antibody-antigen interactions were detected using a chemiluminescent substrate (Merck). To verify equal loading, immunoblots were also probed with an anti-GAPDH monoclonal antibody (Cell Signaling).

MIS Pathway Analysis

Cells were cultured in DMEM F12/1% FBS medium overnight, and then incubated with LRMIS (0-25 nM) at 37° C. for 6 hours. Western blotting was performed using anti-phosphorylated SMAD 1/5, anti-phosphorylated AKT, anti-cleaved caspase 3, anti-cleaved PARP, and anti-GAPDH primary antibodies (1:1.000; Cell Signaling), anti-ALK2, and anti-ALK3 antibodies (1 μg/ml; R&D system) at 4° C. overnight, followed by anti-rabbit and anti-goat IgG HRP secondary antibodies (1:10.000; Sigma) at room temperature for 1 hour.

Clonogenic Survival

Cells were plated in 24-well plates (50 cells/well) in DMEM F12/1% FBS medium overnight. LRMIS (0-25 nM) were then added for 11 days of culture. For COV434-MISRII cells, which grow as clearly individualized clones, colonies were fixed with a methanol/acetic acid solution (3:1) at 4° C. for 20 min, stained with 10% Giemsa, and counted. For SKOV3-MISRII, OVCAR8, KGN cells and cells from patient's ascites, the number of clones was estimated from the confluence area, determined using the Celigo Imaging System after cell staining with Hoechst 33342 trihydrochloride (Invitrogen H1399, 0.25 μg/ml for 15 min).

Apoptosis Assays

Apoptosis initiation was measured using the Caspase-Glos-3/7 assay (Promega). Cells were plated on white 96-well plates and incubated with LRMIS (0-25 nM) for 6 hours. Upon addition of the proluminescent caspase-3/7 DEVD-aminoluciferin substrate, caspase-3/7 generated free aminoluciferin that, consumed by luciferase, produced a luminescent signal proportional to the caspase-3/7 activity. The luminescent signal was quantified 30 min after substrate addition with a PHERASTAR microplate reader.

For a more complete analysis of apoptosis, the Annexin V-FITC Apoptosis Detection Kit (Beckman Coulter IM3614) was used. Approximately 100,000 cells per well were seeded in 24-well plates and incubated or not with 25 nM LRMIS, or 150 nM staurosporin (positive control) for 24 h. Adherent and detached cells were collected and centrifuged at 900 rpm for 5 min. After washes with PBS, cells were stained with 130 μl of a mixture containing 10 μl FITC-labeled annexin V and 20 μl 7AAD in 100 μl annexin buffer on ice in the dark for 15 min. After addition of 400 μl annexin buffer, fluorescence signal data were acquired by flow cytometry within 30 min, and data were analyzed with the Kaluza Flow Analysis software (Beckman Coulter).

Immunofluorescence

For each assay, 30 000 cells were grown on 22-mm square glass coverslips in 35-mm culture dishes in DMEM F12/10% FBS overnight. Cells were then starved with 1% FBS medium for 24 h before incubation with 25 nM LRMIS for 1 h 30. Cells were then fixed in 3.7% paraformaldehyde/PBS for 20 min and permeabilized in acetone at −20° C. for 30 s. Cells were washed twice with PBS/0.1% BSA and incubated with P3X63 (irrelevant antibody) (Köhler et al., 1976), the anti-MISRII 12G4 and anti-ALK2, anti-ALK3, anti-ALK6 (R&D) primary antibodies in the dark for 1 h. After another wash, cells were incubated with goat-FITC-labeled secondary antibodies in PBS/0.1% BSA for 1 h. Then, they were washed three times with PBS/0.1% BSA and once with PBS. Coverslips were mounted with EverBrite™ Hardest Mounting Medium with DAPI (Biotium, Inc., Fremont, Calif.) and analyzed the day after with a Zeiss Axioplan 2 Imaging microscope.

Cell Viability Assay

For cell viability/proliferation testing, the CellTiter 96 AQueous One Solution Cell Proliferation Assay system (Promega) was used according to the manufacturer's instructions. Five thousand cells were plated in each well of a 96-well plate and cultured in 50 μl DMEM F12/1% FBS medium overnight. Cells were then incubated with LRMIS (0-25 nM) for 3 days. Then, 10 μl of CellTiter 96 AQueous One Solution reagent was added per well, and plates were incubated in humidified 5% CO2 atmosphere until the positive control wells became brown (from 1 to 2 h, depending on the cell line). Then, absorbance was measured at 490 nm using a PHERASTAR microplate reader. Three replicate wells were used for each condition.

Statistical Analysis

Statistical analyses concerning differences in caspase-3/7 activity and cell viability/proliferation were performed with the Prism software and ANOVA (Tukey's Multiple Comparison Test).

Results

Recombinant MIS Induces MIS Signaling in COV434-MISRII and SKOV3-MISRII Cells

Before evaluating the involvement of the different MISRIs, we analyzed MIS/MISRII signaling in two MISRII-positive ovarian cancer cell lines: COV434-MISRII (Kersual et al., 2014) and SKOV3-MISRII cells. Indeed, we and other authors found that MISRII expression in cell lines derived from ovarian carcinomas and ovarian carcinoma ascites rapidly and progressively decreases after long-term culture (Estupina et al., 2017; Pépin et al., 2015), thus limiting experiment reproducibility. For all the experiments described in this study, we used human recombinant AMH (LR-AMH; (Pépin et al., 2013)) produced in CHO cells (Evitria AG, Zurich, Switzerland) according to the WO2014/164891 patent (Pépin, 2014) (data not shown). LR-AMH has the advantage of being completely cleaved while being the full-length hormone, thus combining efficiency and stability (Pépin et al., 2013; Wilson et al., 1993). We performed all experiments with LR-AMH in culture medium containing 1% FBS because it was reported that bovine AMH can signal through human AMHRII (Cate et al., 1986). In these experimental conditions, AMH concentration in the medium ranged from 5 to 10 pM in fresh medium to about 10 to 15 pM after 5 days of culture.

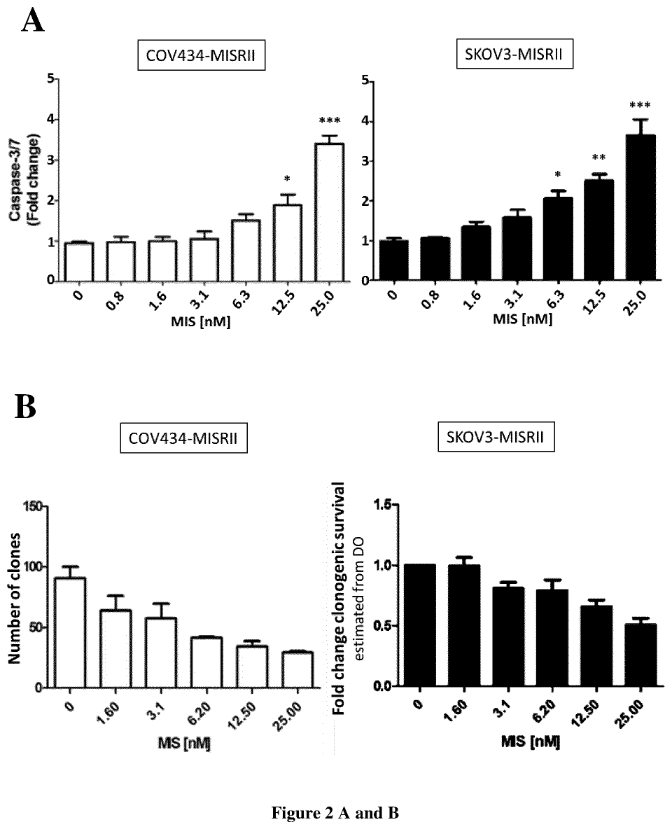

In both cell lines, SMAD1/5 phosphorylation was induced at all tested LRMIS concentrations (from 1.6 to 25 nM). Apoptosis, evaluated by measuring caspase-3/7 activity, was significantly induced starting at 12.5 nM LRMIS in COV434-MISRII cells and at 6.3 nM LRMIS in SKOV3-MISRII cells (FIG. 2A). We confirmed apoptosis induction by western blot analysis of cleaved caspase-3/7 and cleaved PARP (data not shown). Moreover, flow cytometry analysis showed that incubation with 25 nM LRMIS for 24 hours strongly induced apoptosis in COV434-MISRII cells compared with untreated cells (12.5% versus 3.6% of Annexin V-positive cells, and 16.3% versus 5.3% of AnnexinV/7AAD-positive cells), and to a lower extent also in SKOV3-MISRII cells (4.5% versus 5.4% of Annexin V-positive cells, and 11.3% versus 1.7% of AnnexinV/7AAD-positive cells) (data not shown). Finally, at all tested LRMIS concentrations, clonogenic survival was reduced in both cell lines (FIG. 2B). These results confirmed that the COV434-MISRII and SKOV3-MISRII cells are relevant models to study MIS signaling.

In Ovarian Cancer Cells, ALK3 is the Main MISRI Involved in MIS Signaling

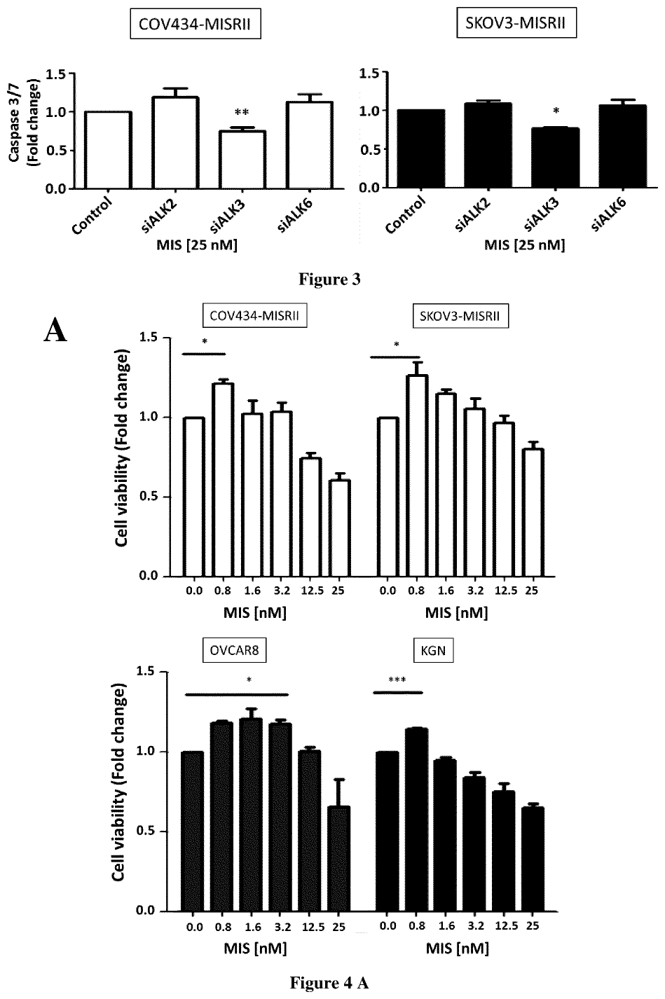

To analyze MISRI involvement in MIS signaling in ovarian cancer cells, we transfected COV434-MISRII and SKOV3-MISRII cells with siRNAs targeting ALK2, ALK3 and ALK6. Due to the role of these receptors in different signaling pathways, their shRNA-mediated silencing was lethal in these cells. PCR and western blot analyses showed that a mixture of three siRNAs against ALK2 (siAlk2) and a mixture of three siRNAs against ALK6 (siAlk6) efficiently inhibited their expression (data not shown). Conversely, ALK3 silencing (siAlk3) was less efficient, particularly in COV434-MISRII cells. Incubation with LRMIS (25 nM, 6 hours) induced SMAD1/5 phosphorylation in siAlk2 and siAlk6, but not in siAlk3 COV434-MISRII and SKOV3-MISRII cells (data not shown). Caspase-3/7 activity and cleavage were not significantly different in siAlk2 and siAlk6 COV434-MISRII and SKOV3-MISRII cells and in COV434-MISRII and SKOV3-MISRII cells transfected with a control siRNA (FIG. 3). Conversely, apoptosis was reduced by about 25% in siAlk3 COV434-MISRII and SKOV3-MISRII cells compared with control. These results were confirmed by western blot analysis of PARP and caspase-3/7 cleavage (data not shown). These findings indicate that, despite incomplete silencing, MIS signaling is reduced mainly in siAlk3 COV434-MISRII and SKOV3-MISRII cells, demonstrating that ALK3 is the favorite MISRI receptor for MIS signaling in ovarian cancer cells.

In Ovarian Cancer Cells, MIS Modulates ALK2 and ALK3 Expression

We then investigated MIS effect on MISRII, ALK2, ALK3 and ALK6 expression in four MISRII-positive ovarian cancer cell lines: COV434-MISRII (sex cord stromal tumor), SKOV3-MISRII (epithelial cancer), OVCAR8 (epithelial cancer), and KGN (granulosa cell tumor). Immunofluorescence (IF) analysis showed that MISRII and ALK2 were clearly expressed in all four cell lines in basal condition (1% FBS corresponding to 10 pM MIS), and their expression was not modulated by incubation with 25 nM LRMIS for 90 min (data not shown). ALK3 expression was not detectable by IF in basal condition, but was induced by MIS addition (data not shown) in all four cell lines. ALK6 was not detectable in both experimental conditions.

Then, to determine the role of ALK2 and ALK3, we assessed their expression and that of MIS signaling proteins by western blotting in basal conditions and after incubation with LRMIS (1.6 to 25 nM) for 6 hours. In all four cell lines (data not shown), ALK2 basal expression decreased upon incubation with LRMIS and was almost undetectable in the presence of 6.25 or 12.5 nM LRMIS. Conversely, ALK3 expression increased upon LRMIS exposure. Moreover, SMAD1/5 phosphorylation caspase-3/7 activity, and caspase 3 and PARP cleavage increased in parallel with ALK3 expression (data not shown).

To analyze the involvement of non-SMAD pathways in MIS signaling (Beck et al., 2016; Zhang, 2017), we monitored AKT phosphorylation and found that it decreased upon incubation with LRMIS, as observed for ALK2 expression (data not shown).

These results confirmed that in ovarian carcinoma cells, ALK3 is the major MISRI in MIS signaling through the SMAD pathway for inducing apoptosis (starting around 6 nM of LRMIS). ALK2 is expressed in basal conditions (around 10 pM MIS) and then its expression is reduced upon incubation with LRMIS.

Cell Survival Promoted by Low MIS Concentrations is Abrogated by siRNA-Mediated MIS Silencing

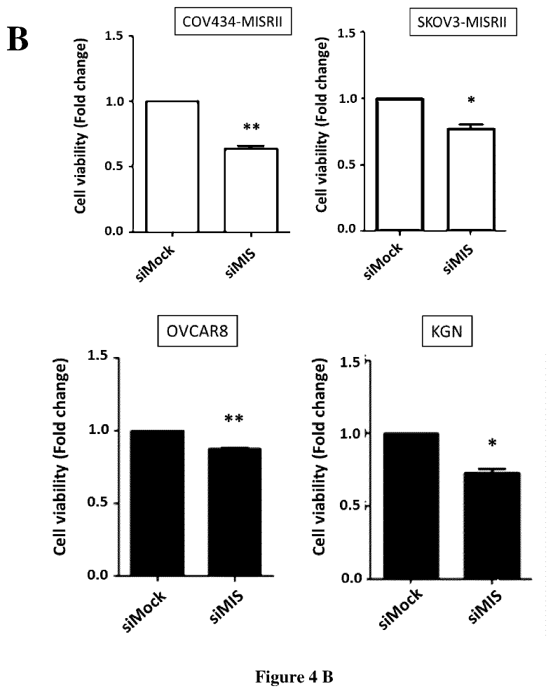

To analyze the effect of MIS concentration on its signaling, we used the MTS assay which is more appropriate to measure viability and proliferation than clonogenic survival assay; this last one being more suitable to detect apoptosis induction by high MIS concentrations (FIG. 2). In the four cell lines, cell viability was increased by the lowest tested LRMIS concentrations (e.g., 0.8 nM LRMIS for KGN, COV34-MISRII and SKOV3-MISRII cells, and up to 3.2 nM LRMIS for OVCAR8 cells), whereas it was reduced by incubation with high LRMIS doses (FIG. 4A). We obtained similar results for AKT phosphorylation (data not shown).

Then, we transfected the four cell lines with siRNAs against MIS. Due to the important MIS production, particularly in COV434-MISRII cells, and despite the use of a pool of endoribonuclease-prepared siRNAs (Kittler et al., 2007), we could not fully silence MIS (data not shown). However, this partial MIS depletion was sufficient to reduce AKT phosphorylation (data not shown) and cell viability by 20% (OVCAR8 cells) to 40% (COV434-MISRII cells) (FIG. 4B).

Discussion

Here, using two ovarian cancer cell lines (COV434-MISRII and SKOV3-MISRII), we found that ALK3 is the favorite MISRI for MIS signaling and apoptosis induction. In four ovarian cancer cell lines (COV434-MISRII, SKOV3-MISRII, OVCAR8 and KGN), we showed that ALK2 and ALK3 are modulated by incubation with LRMIS, and that ALK3 is preferentially expressed when high doses of LRMIS are used to induce apoptosis (FIGS. 2A and 2B). These results, confirmed in tumor cells isolated from ascites samples of two patients with ovarian carcinoma, are currently used to develop new therapeutic strategies.

MIS has been proposed as a potential treatment for gynecologic tumors since 1979 (Donahoe et al., 1979), based on the observation by RE Scully that epithelial ovarian carcinoma resembles histologically the tissues derived from Müllerian ducts (Scully, 1970). Many studies, reviewed by Kim J H et al., validated the potential application of MIS as a bio-drug for cancer therapy (Kim et al., 2014) in ovarian cancer (Anttonen et al., 2011; Fuller et al., 1982; Masiakos et al., 1999; Pieretti-Vanmarcke et al., 2006; Stephen et al., 2002), cervical and endometrial cancer (Barbie et al., 2003; Renaud et al., 2005) as well as in non-Müllerian tumors, such as breast (Gupta et al., 2005) and prostate cancer (Hoshiya et al., 2003). Specifically, these studies showed that high doses of MIS can inhibit cancer cell growth in vitro and in vivo, in cell lines and in patient samples. Interestingly, recent results suggested that MIS could be efficient also in chemotherapy-resistant cancer cells and cancer stem cells (Meirelles et al., 2012; Wei et al., 2010). The major issue for a clinical application of this strategy is the availability of high amount of clinical-grade MIS. To our knowledge, the most advanced strategy is the one developed by Pépin et al. (i.e., LRMIS with an albumin leader sequence and a cleavage site modification leading to high yield of bioactive MIS) (Pépin et al., 2013).

The common point of these studies is that they all used high doses of MIS to treat cancer cells, typically from 25 to 200 nM. This concentration has to be compared to the highest MIS serum concentration observed physiologically (boys from birth to puberty), which is lower than 1 nM (around 50 ng/ml). This is perfectly logical because this strategy is based on MIS induction of apoptosis during Müllerian duct regression. We obtained similar results in the present study, but we also focused on the observation that at low concentration (0.8 nM to 6.1 nM, depending on the cell line; FIG. 4A) MIS promoted cell survival/proliferation.

Moreover, Beck T N et al. showed that in lung cancer, MIS/MISRII signaling regulates epithelial-mesenchymal transition (EMT) and promotes cell survival/proliferation (Beck et al., 2016). They suggested that MIS/MISRII signaling role in EMT regulation was important for chemoresistance. In the present study using anti-MIS siRNAs, we confirmed the involvement of MIS in survival of ovarian carcinoma cells (FIG. 4B).

### REFERENCES

Throughout this application, various references describe the state of the art to which this invention pertains. The disclosures of these references are hereby incorporated by reference into the present disclosure.
Anttonen, M., Färkkilä, A., Tauriala, H., Kauppinen, M., Maclaughlin, D. T., Unkila-Kallio, L., Bützow, R., and Heikinheimo, M. (2011). Anti-Müllerian hormone inhibits growth of AMH type II receptor-positive human ovarian granulosa cell tumor cells by activating apoptosis. Lab. Investig. J. Tech. Methods Pathol. 91, 1605-1614.
Bakkum-Gamez, J. N., Aletti, G., Lewis, K. A., Keeney, G. L., Thomas, B. M., Navarro-Teulon, I., and Cliby, W. A. (2008). Müllerian inhibiting substance type II receptor (MISIIR): a novel, tissue-specific target expressed by gynecologic cancers. Gynecol. Oncol. 108, 141-148.
Barbie, T. U., Barbie, D. A., MacLaughlin, D. T., Maheswaran, S., and Donahoe, P. K. (2003). Mullerian Inhibiting Substance inhibits cervical cancer cell growth via a pathway involving p130 and p107. Proc. Natl. Acad. Sci. U.S.A 100, 15601-15606.
Basal, E., Ayeni, T., Zhang, Q., Langstraat, C., Donahoe, P. K., Pepin, D., Yin, X., Leof, E., and Cliby, W. (2016). Patterns of Müllerian Inhibiting Substance Type II and Candidate Type I Receptors in Epithelial Ovarian Cancer. Curr. Mol. Med. 16, 222-231.
Beck, T. N., Korobeynikov, V. A., Kudinov, A. E., Georgopoulos, R., Solanki, N. R., Andrews-Hoke, M., Kistner, T. M., Pépin, D., Donahoe, P. K., Nicolas, E., et al. (2016). Anti-Müllerian Hormone Signaling Regulates Epithelial Plasticity and Chemoresistance in Lung Cancer. Cell Rep.
Belville, C., Jamin, S. P., Picard, J.-Y., Josso, N., and di Clemente, N. (2005). Role of type I receptors for anti-Müllerian hormone in the SMAT-1 Sertoli cell line. Oncogene 24, 4984-4992.
Bougherara, H., Némati, F., Nicolas, A., Massonnet, G., Pugnière, M., Ngô, C., Le Frère-Belda, M.-A., Leary, A., Alexandre, J., Meseure, D., et al. (2017). The humanized anti-human AMHRII mAb 3C23K exerts an anti-tumor activity against human ovarian cancer through tumor-associated macrophages. Oncotarget 8, 99950-99965.
Cate, R. L., Mattaliano, R. J., Hession, C., Tizard, R., Farber, N. M., Cheung, A., Ninfa, E. G., Frey, A. Z., Gash, D. J., and Chow, E. P. (1986). Isolation of the bovine and human genes for Müllerian inhibiting substance and expression of the human gene in animal cells. Cell 45, 685-698.
Chan-Penebre, E., Armstrong, K., Drew, A., Grassian, A. R., Feldman, I., Knutson, S. K., Kuplast-Barr, K., Roche, M., Campbell, J., Ho, P., et al. (2017). Selective Killing of SMARCA2- and SMARCA4-deficient Small Cell Carcinoma of the Ovary, Hypercalcemic Type Cells by Inhibition of EZH2: In Vitro and In Vivo Preclinical Models. Mol. Cancer Ther. 16, 850-860.
Clarke, T. R., Hoshiya, Y., Yi, S. E., Liu, X., Lyons, K. M., and Donahoe, P. K. (2001). Müllerian inhibiting substance signaling uses a bone morphogenetic protein (BMP)-like pathway mediated by ALK2 and induces SMAD6 expression. Mol. Endocrinol. Baltim. Md. 15, 946-959.
Di Clemente, N., Jamin, S. P., Lugovskoy, A., Carmillo, P., Ehrenfels, C., Picard, J.-Y., Whitty, A., Josso, N., Pepinsky, R. B., and Cate, R. L. (2010). Processing of anti-mullerian hormone regulates receptor activation by a mechanism distinct from TGF-beta. Mol. Endocrinol. Baltim. Md. 24, 2193-2206.
Donahoe, P. K., Swann, D. A., Hayashi, A., and Sullivan, M. D. (1979). Müllerian duct regression in the embryo correlated with cytotoxic activity against human ovarian cancer. Science 205, 913-915.
Donahoe, P. K., Clarke, T., Teixeira, J., Maheswaran, S., and MacLaughlin, D. T. (2003). Enhanced purification and production of Müllerian inhibiting substance for therapeutic applications. Mol. Cell. Endocrinol. 211, 37-42.
Donnell, R. L., McCormick, A., Mukhopadhyay, A., Woodhouse, L. C., Moat, M., Grundy, A., Dixon, M., Kaufman, A., Soohoo, S., Elattar, A., et al. (2014). The use of ovarian cancer cells from patients undergoing surgery to generate primary cultures capable of undergoing functional analysis. PloS One 9, e90604.
Estupina, P., Fontayne, A., Barret, J.-M., Kersual, N., Dubreuil, O., Le Blay, M., Pichard, A., Jarlier, M., Pugniere, M., Chauvin, M., et al. (2017). The anti-tumor efficacy of 3C23K, a glyco-engineered humanized anti-MISRII antibody, in an ovarian cancer model is mainly mediated by engagement of immune effector cells. Oncotarget 8, 37061-37079.
Fuller, A. F., Guy, S., Budzik, G. P., and Donahoe, P. K. (1982). Mullerian inhibiting substance inhibits colony growth of a human ovarian carcinoma cell line. J. Clin. Endocrinol. Metab. 54, 1051-1055.
Gill, S. E., Zhang, Q., Keeney, G. L., Cliby, W. A., and Weroha, S. J. (2017). Investigation of factors affecting the efficacy of 3C23K, a human monoclonal antibody targeting MISIIR. Oncotarget.
Gupta, V., Carey, J. L., Kawakubo, H., Muzikansky, A., Green, J. E., Donahoe, P. K., MacLaughlin, D. T., and Maheswaran, S. (2005). Mullerian inhibiting substance suppresses tumor growth in the C3(1)T antigen transgenic mouse mammary carcinoma model. Proc. Natl. Acad. Sci. U.S.A 102, 3219-3224.
Horbelt, D., Denkis, A., and Knaus, P. (2012). A portrait of Transforming Growth Factor β superfamily signalling: Background matters. Int. J. Biochem. Cell Biol. 44, 469-474. Hoshiya, Y., Gupta, V., Segev, D. L., Hoshiya, M., Carey, J. L., Sasur, L. M., Tran, T. T., Ha, T. U., and Maheswaran, S. (2003). Mullerian Inhibiting Substance induces NFkB signaling in breast and prostate cancer cells. Mol. Cell. Endocrinol. 211, 43-49.
Josso, N., and Clemente, N. di (2003). Transduction pathway of anti-Müllerian hormone, a sex-specific member of the TGF-beta family. Trends Endocrinol. Metab. TEM 14, 91-97.
Josso, N., Racine, C., di Clemente, N., Rey, R., and Xavier, F. (1998). The role of anti-Müllerian hormone in gonadal development. Mol. Cell. Endocrinol. 145, 3-7.
Kersual, N., Garambois, V., Chardès, T., Pouget, J.-P., Salhi, I., Bascoul-Mollevi, C., Bibeau, F., Busson, M., Vie, H., Clémenceau, B., et al. (2014). The human Müllerian inhibiting substance type II receptor as immunotherapy target for ovarian cancer. Validation using the mAb 12G4. MAbs 6, 1314-1326.
Kim, J. H., MacLaughlin, D. T., and Donahoe, P. K. (2014). Müllerian inhibiting substance/anti-Müllerian hormone: A novel treatment for gynecologic tumors. Obstet. Gynecol. Sci. 57, 343-357.
Kittler, R., Surendranath, V., Heninger, A.-K., Slabicki, M., Theis, M., Putz, G., Franke, K., Caldarelli, A., Grabner, H., Kozak, K., et al. (2007). Genome-wide resources of endoribonuclease-prepared short interfering RNAs for specific loss-of-function studies. Nat. Methods 4, 337-344.
Köhler, G., Howe, S. C., and Milstein, C. (1976). Fusion between immunoglobulin-secreting and nonsecreting myeloma cell lines. Eur. J. Immunol. 6, 292-295.
Masiakos, P. T., MacLaughlin, D. T., Maheswaran, S., Teixeira, J., Fuller, A. F., Shah, P. C., Kehas, D. J., Kenneally, M. K., Dombkowski, D. M., Ha, T. U., et al. (1999). Human ovarian cancer, cell lines, and primary ascites cells express the human Mullerian inhibiting substance (MIS) type II receptor, bind, and are responsive to MIS. Clin. Cancer Res. Off. J. Am. Assoc. Cancer Res. 5, 3488-3499.
Mazumder, S., Johnson, J. M., Swank, V., Dvorina, N., Martelli, E., Ko, J., and Tuohy, V. K. (2017). Primary Immunoprevention of Epithelial Ovarian Carcinoma by Vaccination against the Extracellular Domain of Anti-Müllerian Hormone Receptor II. Cancer Prev. Res. Phila. Pa. 10, 612-624.
Meirelles, K., Benedict, L. A., Dombkowski, D., Pepin, D., Preffer, F. I., Teixeira, J., Tanwar, P. S., Young, R. H., MacLaughlin, D. T., Donahoe, P. K., et al. (2012). Human ovarian cancer stem/progenitor cells are stimulated by doxorubicin but inhibited by Mullerian inhibiting substance. Proc. Natl. Acad. Sci. U.S.A 109, 2358-2363.
Nachtigal, M. W., and Ingraham, H. A. (1996). Bioactivation of Müllerian inhibiting substance during gonadal development by a kex2/subtilisin-like endoprotease. Proc. Natl. Acad. Sci. U.S.A 93, 7711-7716.
Nishi, Y., Yanase, T., Mu, Y., Oba, K., Ichino, I., Saito, M., Nomura, M., Mukasa, C., Okabe, T., Goto, K., et al. (2001). Establishment and characterization of a steroidogenic human granulosa-like tumor cell line, KGN, that expresses functional follicle-stimulating hormone receptor. Endocrinology 142, 437-445.
Orvis, G. D., Jamin, S. P., Kwan, K. M., Mishina, Y., Kaartinen, V. M., Huang, S., Roberts, A. B., Umans, L., Huylebroeck, D., Zwijsen, A., et al. (2008). Functional redundancy of TGF-beta family type I receptors and receptor-Smads in mediating anti-Mullerian hormone-induced Mullerian duct regression in the mouse. Biol. Reprod. 78, 994-1001.
Pellatt, A. J., Mullany, L. E., Herrick, J. S., Sakoda, L. C., Wolff, R. K., Samowitz, W. S., and Slattery, M. L. (2018). The TGFβ-signaling pathway and colorectal cancer: associations between dysregulated genes and miRNAs. J. Transl. Med. 16, 191.
Pépin, D. (2014). Modified mullerian inhibiting substance (mis) proteins and uses thereof for the treatment of diseases.
Pépin, D., Hoang, M., Nicolaou, F., Hendren, K., Benedict, L. A., Al-Moujahed, A., Sosulski, A., Marmalidou, A., Vavvas, D., and Donahoe, P. K. (2013). An albumin leader sequence coupled with a cleavage site modification enhances the yield of recombinant C-terminal Mullerian Inhibiting Substance. Technol. Elmsford N 1, 63-71.
Pépin, D., Sosulski, A., Zhang, L., Wang, D., Vathipadiekal, V., Hendren, K., Coletti, C. M., Yu, A., Castro, C. M., Birrer, M. J., et al. (2015). AAV9 delivering a modified human Mullerian inhibiting substance as a gene therapy in patient-derived xenografts of ovarian cancer. Proc. Natl. Acad. Sci. U.S.A 112, E4418-4427.
Philibert, P., Stoessel, A., Wang, W., Sibler, A.-P., Bec, N., Larroque, C., Saven, J. G., Courtete, J., Weiss, E., and Martineau, P. (2007). A focused antibody library for selecting scFvs expressed at high levels in the cytoplasm. BMC Biotechnol. 7, 81.
Pieretti-Vanmarcke, R., Donahoe, P. K., Szotek, P., Manganaro, T., Lorenzen, M. K., Lorenzen, J., Connolly, D. C., Halpern, E. F., and MacLaughlin, D. T. (2006). Recombinant human Mullerian inhibiting substance inhibits long-term growth of MIS type II receptor-directed transgenic mouse ovarian cancers in vivo. Clin. Cancer Res. Off. J. Am. Assoc. Cancer Res. 12, 1593-1598.
Rehman, Z. U., Worku, T., Davis, J. S., Talpur, H. S., Bhattarai, D., Kadariya, I., Hua, G., Cao, J., Dad, R., Farmanullah, null, et al. (2017). Role and mechanism of AMR in the regulation of Sertoli cells in mice. J. Steroid Biochem. Mol. Biol. 174, 133-140.
Renaud, E. J., MacLaughlin, D. T., Oliva, E., Rueda, B. R., and Donahoe, P. K. (2005). Endometrial cancer is a receptor-mediated target for Mullerian Inhibiting Substance. Proc. Natl. Acad. Sci. U.S.A 102, 111-116.
Robin, G., and Martineau, P. (2012). Synthetic customized scFv libraries. Methods Mol. Biol. Clifton N.J. 907, 109-122.
Salhi, I., Cambon-Roques, S., Lamarre, I., Laune, D., Molina, F., Pugnière, M., Pourquier, D., Gutowski, M., Picard, J.-Y., Xavier, F., et al. (2004). The anti-Müllerian hormone type II receptor: insights into the binding domains recognized by a monoclonal antibody and the natural ligand. Biochem. J. 379, 785-793.
Scully, R. E. (1970). Recent progress in ovarian cancer. Hum. Pathol. 1, 73-98.
Sèdes, L., Leclerc, A., Moindjie, H., Cate, R. L., Picard, J.-Y., di Clemente, N., and Jamin, S. P. (2013). Anti-Müllerian hormone recruits BMPR-IA in immature granulosa cells. PloS One 8, e81551.
Song, J. Y., Chen, K. Y., Kim, S. Y., Kim, M. R., Ryu, K. S., Cha, J. H., Kang, C. S., MacLaughlin, D. T., and Kim, J. H. (2009). The expression of Müllerian inhibiting substance/anti-Müllerian hormone type II receptor protein and mRNA in benign, borderline and malignant ovarian neoplasia. Int. J. Oncol. 34, 1583-1591.
Stephen, A. E., Pearsall, L. A., Christian, B. P., Donahoe, P. K., Vacanti, J. P., and MacLaughlin, D. T. (2002). Highly purified müllerian inhibiting substance inhibits human ovarian cancer in vivo. Clin. Cancer Res. Off. J. Am. Assoc. Cancer Res. 8, 2640-2646.
Uhlen, M., Zhang, C., Lee, S., Sjöstedt, E., Fagerberg, L., Bidkhori, G., Benfeitas, R., Arif, M., Liu, Z., Edfors, F., et al. (2017). A pathology atlas of the human cancer transcriptome. Science 357.
Visser, J. A., Olaso, R., Verhoef-Post, M., Kramer, P., Themmen, A. P., and Ingraham, H. A. (2001). The serine/threonine transmembrane receptor ALK2 mediates Müllerian inhibiting substance signaling. Mol. Endocrinol. Baltim. Md. 15, 936-945.
Wei, X., Dombkowski, D., Meirelles, K., Pieretti-Vanmarcke, R., Szotek, P. P., Chang, H. L., Preffer, F. I., Mueller, P. R., Teixeira, J., MacLaughlin, D. T., et al. (2010). Müllerian inhibiting substance preferentially inhibits stem/progenitors in human ovarian cancer cell lines compared with chemotherapeutics. Proc. Natl. Acad. Sci. U.S.A 107, 18874-18879.
Wilson, C. A., di Clemente, N., Ehrenfels, C., Pepinsky, R. B., Josso, N., Vigier, B., and Cate, R. L. (1993). Mullerian inhibiting substance requires its N-terminal domain for maintenance of biological activity, a novel finding within the transforming growth factor-beta superfamily. Mol. Endocrinol. Baltim. Md. 7, 247-257.
Yuan, Q., Simmons, H. H., Robinson, M. K., Russeva, M., Marasco, W. A., and Adams, G. P. (2006). Development of engineered antibodies specific for the Müllerian inhibiting substance type II receptor: a promising candidate for targeted therapy of ovarian cancer. Mol. Cancer Ther. 5, 2096-2105.
Yuan, Q.-A., Robinson, M. K., Simmons, H. H., Russeva, M., and Adams, G. P. (2008). Isolation of anti-MISIIR scFv molecules from a phage display library by cell sorter biopanning. Cancer Immunol. Immunother. CII 57, 367-378.
Zhan, Y., Fujino, A., MacLaughlin, D. T., Manganaro, T. F., Szotek, P. P., Arango, N. A., Teixeira, J., and Donahoe, P. K. (2006). Müllerian inhibiting substance regulates its receptor/SMAD signaling and causes mesenchymal transition of the coelomic epithelial cells early in Müllerian duct regression. Dev. Camb. Engl. 133, 2359-2369.
Zhang, Y. E. (2017). Non-Smad Signaling Pathways of the TGF-β Family. Cold Spring Harb. Perspect. Biol. 9.
Zhang, H., Vollmer, M., De Geyter, M., Litzistorf, Y., Ladewig, A., Dürrenberger, M., Guggenheim, R., Miny, P., Holzgreve, W., and De Geyter, C. (2000). Characterization of an immortalized human granulosa cell line (COV434). Mol. Hum. Reprod. 6, 146-153.

1. A method of treating a müllerian inhibiting substance (MIS) or a Müllerian Inhibiting Substance type II receptor (MISRII) positive cancer in a subject in need thereof, comprising administering to the subject a therapeutically effective amount of a MIS inhibitor.

2. The method according to claim 1, wherein the MIS inhibitor is an expression inhibitor.

3. The method according to claim 1, wherein the MIS inhibitor is an activity inhibitor.

4. The method according to claim 2 wherein said expression inhibitor is an antisense oligonucleotides, siRNA and/or a ribozymes.

5. The method according to claim 3 wherein said activity inhibitor is an antibody, a peptide, a polypeptide, an aptamer or a small organic molecule.

6. The method according to claim 1, wherein the MSI inhibitor blocks recruiting of an MIS type I receptor (MISRI) by the complex MISRII/MIS.

7. The method according to claim 1, wherein the MIS or MISRII positive cancer is selected from the group consisting of lung cancer, colorectal cancer and gynecological cancer.

8. The method according to claim 7, wherein the MIS or MISRII positive cancer is a gynecological cancer.

9. The method according to claim 8, wherein the gynecological cancer is an ovarian cancer.

10. (canceled)

11. The method according to claim 6, wherein the MISRI is ALK2, ALK3 or ALK6.
